(19) United States
(12) Patent Application Publication
(10) Pub. No.: US 2009/0028747 A1
(43) Pub. Date: Jan. 29, 2009

(54) METHOD FOR USING A STERILE PRODUCT PREPARATION FOR SURGICAL INSTRUMENTS

(75) Inventor: Nikolai P. Burkart, Wallhausen (DE)

Correspondence Address: BACHMAN & LAPOINTE, P.C., 900 CHAPEL STREET, SUITE 1201, NEW HAVEN, CT, 06510, US

(73) Assignee: BURKART BETEILIGUNGEN GMBH, Singen (DE)

(21) Appl. No.: 11/577,123

(22) PCT Filed: Nov. 11, 2004
(86) PCT No.: PCT/EP04/12804

§ 371 Date: May 31, 2007

(30) Foreign Application Priority Data
Oct. 15, 2004 (DE) 10 2004 050 577.2

(51) Int. Cl. A61L 2/00

(52) U.S. Cl. 422/1

(57) ABSTRACT

A method for using a sterile product preparation for surgical instruments or sterile product units of any type during which surgical instruments or sterile product units of the most varied type and origin are fed to a sterile product processing system. The aim of the invention is to provide each surgical instrument or each sterile product unit with a transporter in order to assign the surgical instrument, after having been treated in the sterile product processing system, to a specified user.

The invention relates to a method for operating a sterile product processing system for surgical instruments or sterile product units of any type, in which surgical instruments or sterile product units of the most varied type and origin are fed to a sterile product processing installation, and it relates also to a surgical instrument and a sterile product unit.

In conventional methods for operating a sterile product processing system, surgical instruments and sterile product units are fed to a central sterile product processing system or sterile product processing installation, are sterilized after preliminary cleaning, and are then packaged in a sterile state and then manually dispatched back to the individual departments, the individual operating theaters or the individual medical practices.

Errors frequently arise in this procedure, leading to delays in supplying the surgical instruments and sterile product units to the respective operating theaters or medical practices.

Moreover, high costs are required for the personnel who ensure that the equipment or surgical instruments or sterile product units sterilized in the sterile product processing system or sterile product processing installation are sorted out and manually sent back to the respective departments or operating theaters or medical practices, etc.

The object of the present invention is therefore to make available a method of the type mentioned at the outset, and to make available a sterile product unit and a surgical instrument which avoid the stated disadvantages and with which it is possible, in a simple and cost-effective manner, to optimize a central sterile product processing system and additionally permit tracking of the individual surgical instruments and/or sterile product units.

This object is achieved by the features of patent claim 1 and those of independent patent claim 9.

In the present invention, it has proven particularly advantageous to monitor and optimize a complete process preferably during the sterile product treatment of surgical instruments and/or sterile product units.

It has proven particularly advantageous to provide each surgical instrument or each sterile product unit with at least one transponder that contains user-specific data, such as department, attribution to the medical practice or attribution to the corresponding operating theater. In addition, this transponder contains readable data concerning purchase costs of the surgical instrument, repair costs, exchanged replacement parts, repair interval, depreciation, etc.

These data can be read in by a transmitter, updated and evaluated for example in the area of the sterile product processing by means of a PC, and suitable localization of the surgical instrument is also possible. It should also be noted that this permits suitable stocktaking, management of parts, and equipment management.

It has also proven particularly advantageous that this permits automated sorting of surgical instruments and/or sterile product units after passage through a sterile product processing installation, such that the corresponding sterilized surgical instruments and sterile product units can be returned then to each operating theater in a very cost-effective manner.

This ensures complete tracking of the individual surgical instruments and sterile product units, preferably in the area of the central sterile product processing system, thus affording considerable savings in logistical terms. Transport costs and sorting costs can be saved, maintenance intervals can be checked, which serves for operational safety and ensures the technical state of the surgical instruments.

It also lies within the context of the present invention that corresponding transmitters can be assigned to the sterile product processing installation so as to not only read out data, but also actively input updated data and store these in the transponders of the surgical instruments or the sterile product units. It is also intended to lie within the context of the present invention that the corresponding transmitter can be provided in the area of the central sterile product processing system, for example in the area of a conveyor belt, then communicating with a suitable evaluation computer or the like, for example in order to monitor and control the central sterile product processing system and carry out automated distribution.

Other advantages, features and details of the invention will become clear from the following description of preferred illustrative embodiments and from the drawing, in which:

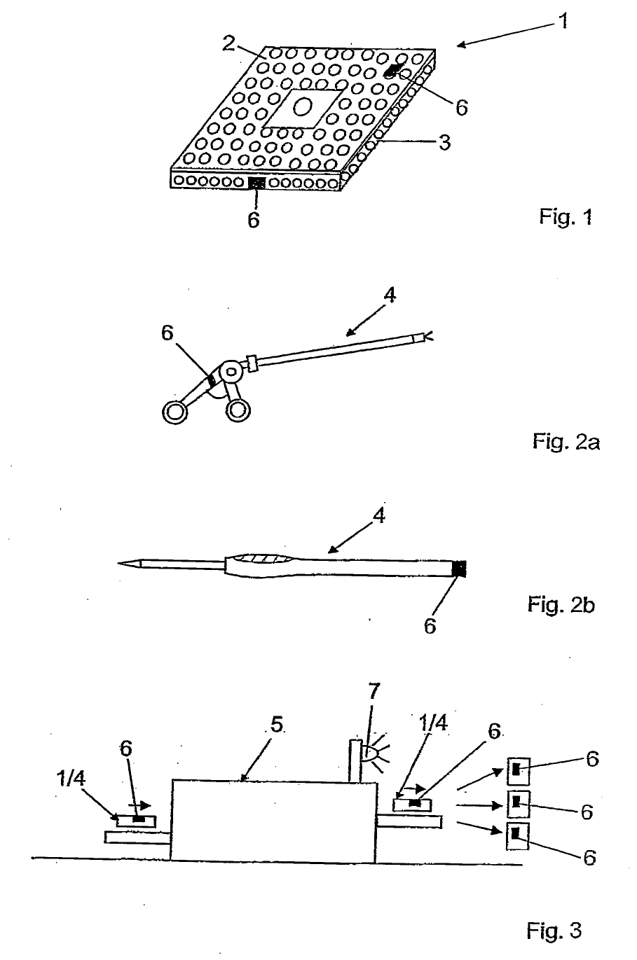

FIG. 1 shows a schematic perspective plan view of a sterile product unit for receiving a plurality of surgical instruments;

FIG. 2a shows a schematic plan view of a surgical instrument;

FIG. 2b shows a schematic plan view of a surgical instrument designed as a scalpel;

FIG. 3 shows a schematic plan view of a sterile product processing installation for processing surgical instruments and/or sterile product units containing surgical instruments.

According to FIG. 1, a sterile product unit 1 is shown which is formed like a box, made up of a lid 2 and a collection container 3. The sterile product units 1 are used to receive surgical instruments 4, for example the ones which are shown schematically in FIGS. 2a and 2b and which can be received in the sterile product unit 1. The conventional sterile product units 1, also called sieves, are used to receive a specific number of selected surgical instruments.

For use in the operating theater, these surgical instruments are first sterilized in a sterile product processing installation 5 indicated in FIG. 3 and are then returned in a sterilized condition to the operating theater for use in an operation.

The surgical instruments 4 can also be of any desired shape and type, as indicated, for example, by the surgical forceps in FIG. 2a or by the scalpel in FIG. 2b. Trocars, endoscopic devices, endoscopic forceps, etc., can fall under the generic term of surgical instrument and can be treated and sterilized in the sterile product processing installation 5.

It is becoming more and more common for a number of hospital departments or a number of hospitals to share a central location for sterile product processing or a sterile product processing installation, and this may, if appropriate, also be located at a separate site from the hospital or operating theater.

For the complete process of sterilization of conventional sterile product units containing surgical instruments, or of used surgical instruments, these have to be sorted out again after sterilization—which takes up a lot of work and time—and then be delivered to the respective departments, surgical departments or operating theaters.

To ensure this logistical process, and other advantages too, it has proven particularly advantageous in the present invention to assign a transponder 6 to the sterile product unit 1 and/or to the surgical instrument 4, in which transponder 6 a large amount of readable data is stored or can be permanently stored anew.

The transponder 6 in the sterile product unit 1 can be assigned to the lid 2 and/or to the collection container 3.

The data relating to the owner or user, to the department, to the operating theater, to the medical practice or the like, are stored in the transponder 6, thus permitting automated assignment or provision or delivery, after sterilization, to the correct location or the correct department or the correct operating theater, possibly also with partial automation.

An optimized equipment management of the surgical instruments 4 and of the sterile product units 1 is also possible, in which, for each surgical instrument 4 for example, the purchase data or age, the purchase costs, the depreciation, wear, repair costs, repair frequency, replacement parts can be stored. In this way, it is possible to generate an exact repair management and a maintenance interval, and a depreciation (for wear and tear) can also be exactly defined by inputting via a central computer, for example in the area of the sterile product processing installation 5.

It is additionally possible, using suitable transmitters 7, to locate the sterile product units 1 and surgical instruments 4, to supplement the data or to retrieve data.

In this way, it is possible for a maintenance interval to be indicated actively for each surgical instrument, such that they always correspond to the optimal application conditions and a defect can be ruled out.

It has also proven advantageous, particularly regarding the sterilization of sterile product units 1 and/or surgical instruments 4, that the transponder 6 stores data on when they have been sterilized in the sterile product processing installation 5. It is then possible, by reading the data in the transponder 6, to exactly determine to which department, to which operating theater or to which medical practice the sterilized surgical instrument 4 and/or the sterilized sterile product unit 1 has to be transported. This very rapidly permits automated selection and sorting, and the transport to the selected operating theaters can also be automated or optimized, for example several sterilized sterile product units 1 and/or surgical instruments 4 for the same operating theater or for the same medical practice can then be delivered there in a common shipment.

It is important, in the present invention, that complete equipment management and tracking of the individual parts or sterile product units 1 and surgical instruments 4 are also permitted in the area of a central sterile product processing installation 5.

### List of Reference Numbers

1 sterile product unit

2 lid

3 collection container

4 surgical instrument

5 sterile product processing installation

6 transponder

7 transmitter

1. A method for operating a sterile product processing system for surgical instruments (4) or sterile product units (1) of any type, in which surgical instruments (4) or sterile product units (1) of the most varied type and origin are fed to a sterile product processing installation (5),
characterized in that
each surgical instrument (4) or each sterile product unit (1) is provided with a transponder (6) in order to assign the surgical instrument (4), after treatment in the sterile product processing installation (5), to a specified user.

2. The method as claimed in claim 1, characterized in that, by assigning a transponder (6) to the sterile product unit (1) or a sieve for receiving a certain unit and selection of surgical instruments (4) or to each surgical instrument (4) itself, it is possible, in a sorting facility arranged downstream of the sterile product processing system, to perform a corresponding evaluation and user assignment.

3. The method as claimed in claim 2, characterized in that, in a first method step, the surgical instruments (4) and/or sterile product units (1) are fed to the sterile product processing installation (5), and a first rough preliminary cleaning takes place.

4. The method as claimed in claim 3, characterized in that assigning transponders (6) to sterile product units (1) and/or to desired surgical instruments (4) allows a selection to be made for different users, for example different hospitals, different surgical departments, different physicians, etc., and this selection allows a corresponding local distribution and assignment of the surgical instruments (4) or of the sterile product units (1) to be carried out in an automated manner.

5. The method as claimed in claim 3, characterized in that assigning a transponder (6) to the surgical instrument (4) and/or to the sterile product unit (1) makes it possible to store specific data for the surgical instrument (4) or for the sterile product unit (1), including data concerning the contents of the latter, in which case data such as age, wear, required repair costs, replacement parts and repaired parts, repairs, and repair management are stored on the transponder (6), read in and/or updated.

6. The method as claimed in claim 3, characterized in that assigning the transponder (6) to the surgical instrument (4) and/or to the sterile product unit (1) permits permanent localization and position determination, by means of which it is possible to determine or retrieve not only the location of the surgical instrument (4) or sterile product unit (1), but also the time at which the surgical instrument (4) or sterile product unit (1) will be available to the user.

7. The method as claimed in claim 3, characterized in that assigning a transponder (6) to the surgical instrument (4) and/or to the sterile product unit (1) permits equipment management, for example for operating theaters, mobile military hospitals, surgical departments, physicians, or the like.

8. The method as claimed in claim 4, characterized in that all the data for the surgical instrument (4) and/or for the sterile product unit (1) are retrieved via corresponding transmitters (7) in the area of the sterile product processing system, in the area of the sterile product packaging, in the area of the sterile product supply and of the sterile product transport, and in the area of the stockroom, and also in the area of the operating theater, field hospital, or the like, and can be stored via a central computer.

9. A surgical instrument (4), or a sterile product unit (1) for receiving a number of selected surgical instruments (4), characterized in that at least one transponder (6) is assigned to the surgical instrument (4) and/or to the sterile product unit (1).

10. The surgical instrument (4) or sterile product unit (1) as claimed in claim 9, characterized in that the transponder (6) is assigned to the surgical instrument (4) particularly in a handle area.

11. The surgical instrument (4) or sterile product unit (1) as claimed in claim 9, characterized in that the transponder (6) is assigned to the sterile product unit (1), particularly to the housing and/or to the lid (2).

12. The surgical instrument (4) or sterile product unit (1) as claimed in claim 9, characterized in that position data, repair data, purchase costs, depreciation, wear, age, size, repair intervals or the like can be retrieved from and/or stored in the transponder (6).

13. The surgical instrument (4) or sterile product unit (1) as claimed in claim 9, characterized in that, after a processing operation in a sterile product processing installation (5), the transponder (6) permits automated sorting and assignment and, if appropriate, transport to the user or stockroom.
